SEQUENCE LISTING (30 sequences)

SEQ ID NO: 1 - DNA, 320 bp, Cuphea palustris
atggtggctgccgcagcaagtgctgcattcttctccgtcgcaaccccgcgaacaaacatttcgccatcgagcttgagcgtccccttcaagcccaaatcaaaccacaatggtggctttcaggttaaggcaaacgccagtgcccatcctaaggctaacggttctgcagtaagtctaaagtctggcagcctcgagactcaggaggacaaaacttcatcgtcgtcccctcctcctcggactttcattaaccagttgcccgtctgggtatgcttctgtctgcagtcacgactgtcttcggggtggctgagaagcagtggccaatg

SEQ ID NO: 2 - protein, 107 aa, Cuphea palustris
MVAAAASAAFFSVATPRTNISPSSLSVPFKPKSNHNGGFQVKANASAHPKANGSAVSLKSGSLETQEDKTSSSSPPPRTFINQLPVWSMLLSAVTTVFGVAEKQWPM

SEQ ID NO: 3 - DNA, 360 bp, Cuphea palustris
  CDS (1)..(360)
atggtggctgctgcagcaagttctgcatgcttccctgttccatccccaggagcctcccctaaacctgggaagttaggcaactggtcatcgagtttgagcccttccttgaagcccaagtcaatccccaatggcggatttcaggttaaggcaaatgccagtgcgcatcctaaggctaacggttctgcagtaactctaaagtctggcagcctcaacactcaggaggacactttgtcgtcgtcccctcctccccgggctttttttaaccagttgcctgattggagtatgcttctgactgcaatcacaaccgtcttcgtggcaccagagaagcggtggactatgtttgataggaaatctaagagg

SEQ ID NO: 4 - protein, 120 aa, Cuphea palustris
MVAAAASSACFPVPSPGASPKPGKLGNWSSSLSPSLKPKSIPNGGFQVKANASAHPKANGSAVTLKSGSLNTQEDTLSSSPPPRAFFNQLPDWSMLLTAITTVFVAPEKRWTMFDRKSKR

SEQ ID NO: 5 - DNA, 436 bp, Cuphea hookeriana
  CDS (1)..(435)
atgttgaagctttcttgcaatgccgccaccgaccagattctgtcgtcggccgtggctcaaaccgcattatggggtcaacccagaaacagatccttttcaatgtccgcccggagaaggggagccgtttgctgcgcgcctccagctgctggaaagccccctgccatgaccgctgttatcccaaaagacggggtggcctcgtccgggtccggcagcctggccgaccagctgaggctcgggagccgtacgcagaatgggctgtcgtacacggagaagttcattgtcaggtgctacgaggtcggtattaacaagacagccactgtcgaaaccatggccaatctcttgcaggaagtaggttgtaaccatgctcagagtgttggattctcaactgacgggtttgcgacgacgcctaccatgaggaaattgaatctgatatggg

SEQ ID NO: 6 - protein, 145 aa, Cuphea hookeriana
MLKLSCNAATDQILSSAVAQTALWGQPRNRSFSMSARRRGAVCCAPPAAGKPPAMTAVIPKDGVASSGSGSLADQLRLGSRTQNGLSYTEKFIVRCYEVGINKTATVETMANLLQEVGCNHAQSVGFSTDGFATTPTMRKLNLIW

SEQ ID NO: 7 - DNA, 357 bp, Cuphea hookeriana
  CDS (1)..(357)
atggtggctaccgctgcaagttctgcattcttccccctcccatccgccgacacctcatcgagacccggaaagctcggcaataagccatcgagcttgagccccctcaagcccaaatcgacccccaatggcggtttgcaggttaaggcaaatgccagtgcccctcctaagatcaatggttccccggtcggtctaaagtcgggcggtctcaagactcaggaagacgctcattcggcccctcctccgcgaacttttatcaaccagttgcctgattggagtatgcttcttgctgcaatcacgactgtcttcttggctgcagagaagcaatggatgatgcttgattggaaacctaagaggcct

SEQ ID NO: 8 - protein, 119 aa, Cuphea hookeriana
MVATAASSAFFPLPSADTSSRPGKLGNKPSSLSPLKPKSTPNGGLQVKANASAPPKINGSPVGLKSGGLKTQEDAHSAPPPRTFINQLPDWSMLLAAITTVFLAAEKQWMMLDWKPKRP

SEQ ID NO: 9 - DNA, 357 bp, Cuphea hookeriana
  CDS (1)..(357)
atggtggctaccgctgcaagctctgcattcttccccgtgtcgtccccggtcacctcctctagaccaggaaagcccggaaatgggtcatcgagcttcagccccatcaagcccaaatttgtcgccaatggcgggttgcaggttaaggcaaacgccagtgcccctcctaagatcaatggttcctcggtcggtctaaagtcctgcagtctcaagactcaggaagacactccttcggcccctgctccacggacttttatcaaccagttgcctgattggagtatgcttcttgctgcaattactactgtcttcttggcagcagagaagcagtggatgatgcttgattggaaacctaagaggcct

SEQ ID NO: 10 - protein, 119 aa, Cuphea hookeriana
MVATAASSAFFPVSSPVTSSRPGKPGNGSSSFSPIKPKFVANGGLQVKANASAPPKINGSSVGLKSCSLKTQEDTPSAPAPRTFINQLPDWSMLLAAITTVFLAAEKQWMMLDWKPKRP

SEQ ID NO: 11 - DNA, 333 bp, Cuphea hookeriana
  CDS (1)..(333)
atggtggctgctgcagcaagttccgcattcttccctgttccagccccgggagcctcccctaaacccgggaagttcggaaattggccctcgagcttgagcccttccttcaagcccaagtcaatccccaatggcggatttcaggttaaggcaaatgacagcgcccatccaaaggctaacggttctgcagttagtctaaagtctggcagcctcaacactcaggaggacacttcgtcgtcccctcctcctcggactttccttcaccagttgcctgattggagtaggcttctgactgcaatcacgaccgtgttcgtgaaatctaagaggcctgacatg

SEQ ID NO: 12 - protein, 111 aa, Cuphea hookeriana
MVAAAASSAFFPVPAPGASPKPGKFGNWPSSLSPSFKPKSIPNGGFQVKANDSAHPKANGSAVSLKSGSLNTQEDTSSSPPPRTFLHQLPDWSRLLTAITTVFVKSKRPDM

SEQ ID NO: 13 - DNA, 270 bp, Cuphea hookeriana
  CDS (1)..(270)
atggtggctgccgcagcaagttctgcattcttctccgttccaaccccgggaatctcccctaaacccgggaagttcggtaatggtggctttcaggttaaggcaaacgccaatgcccatcctagtctaaagtctggcagcctcgagactgaagatgacacttcatcgtcgtcccctcctcctcggactttcattaaccagttgcccgactggagtatgcttctgtccgcaatcacgactatcttcggggcagctgagaagcagtggatgatg

SEQ ID NO: 14 - protein, 90 aa, Cuphea hookeriana
MVAAAASSAFFSVPTPGISPKPGKFGNGGFQVKANANAHPSLKSGSLETEDDTSSSSPPPRTFINQLPDWSMLLSAITTIFGAAEKQWMM

SEQ ID NO: 15 - DNA, 174 bp, Pisum sativum
  CDS (1)..(174)
atggcttcctcagttctttcctctgcagcagttgccacccgcagcaatgttgctcaagctaacatggttgcacctttcactggccttaagtcagctgcctcattccctgtttcaaggaagcaaaaccttgacatcacttccattgccagcaacggcggaagagtgcaatgcagg

SEQ ID NO: 16 - protein, 58 aa, Pisum sativum
MASSVLSSAAVATRSNVAQANMVAPFTGLKSAASFPVSRKQNLDITSIASNGGRVQCR

SEQ ID NO: 17 - DNA, 1242 bp, Cuphea hookeriana
  CDS (1)..(1242)
atggtggctaccgctgcaagctctgcattcttccccgtgtcgtccccggtcacctcctctagaccaggaaagcccggaaatgggtcatcgagcttcagccccatcaagcccaaatttgtcgccaatggcgggttgcaggttaaggcaaacgccagtgcccctcctaagatcaatggttcctcggtcggtctaaagtcctgcagtctcaagactcaggaagacactccttcggcccctgctccacggacttttatcaaccagttgcctgattggagtatgcttcttgctgcaattactactgtcttcttggcagcagagaagcagtggatgatgcttgattggaaacctaagaggcctgacatgcttgtggacccgttcggattgggaagtattgtccagcatgggcttgtgttcaggcagaatttttcgattaggtcctatgaaataggcgctgatcgcactgcgtctatagagacggtgatgaaccacttgcaggaaacggctctcaatcatgttaagagtgcggggcttatgaatgacggctttggtcgtactcctgagatgtataaaaaggaccttatttgggttgtcgcgaaaatgcaggtcatggttaaccgctatcctacttggggtgacacagttgaagtgaatacttgggttgccaagtcagggaaaaatggtatgcgtcgtgattggctcataagtgattgtaatacaggagaaattcttactagagcatcaagcgtgtgggtcatgatgaatcaaaagacaagaagattgtcaaaaattccagatgaggttcggcatgagattgagcctcattttgtggactctcctcccgtcattgaagacgatgaccgaaaacttcccaagctggatgacaagactgctgactccatccgcaagggtctaactccgaagtggaatgacttggatgtcaatcagcacgtcaacaacgtgaagtacatcgggtggattcttgagagtactccacaagaagttctggagacccaggagctatgttcccttaccctggaatacaggcgggaatgcggaagggagagcgtgctggagtccctcactgctgcggacccctctggaaagggctttgggtcccagttccagcaccttctgaggcttgaggatggaggggagattgtgaaggggagaactgagtggcgaccaaagactgcaggtatcaatggggcgataccatccggggagacctcacctggagactct

SEQ ID NO: 18 - protein, 414 aa, Cuphea hookeriana
MVATAASSAFFPVSSPVTSSRPGKPGNGSSSFSPIKPKFVANGGLQVKANASAPPKINGSSVGLKSCSLKTQEDTPSAPAPRTFINQLPDWSMLLAAITTVFLAAEKQWMMLDWKPKRPDMLVDPFGLGSIVQHGLVFRQNFSIRSYEIGADRTASIETVMNHLQETALNHVKSAGLMNDGFGRTPEMYKKDLIWVVAKMQVMVNRYPTWGDTVEVNTWVAKSGKNGMRRDWLISDCNTGEILTRASSVWVMMNQKTRRLSKIPDEVRHEIEPHFVDSPPVIEDDDRKLPKLDDKTADSIRKGLTPKWNDLDVNQHVNNVKYIGWILESTPQEVLETQELCSLTLEYRRECGRESVLESLTAADPSGKGFGSQFQHLLRLEDGGEIVKGRTEWRPKTAGINGAIPSGETSPGDS

SEQ ID NO: 19 - DNA, 1182 bp, Cuphea hookeriana
  CDS (1)..(1182)
atggtggctgccgcagcaagttctgcattcttctccgttccaaccccgggaatctcccctaaacccgggaagttcggtaatggtggctttcaggttaaggcaaacgccaatgcccatcctagtctaaagtctggcagcctcgagactgaagatgacacttcatcgtcgtcccctcctcctcggactttcattaaccagttgcccgactggagtatgcttctgtccgcaatcacgactatcttcggggcagctgagaagcagtggatgatgcttgataggaaatctaagagacccgacatgctcatggaaccgtttggggttgacagtattgttcaggatggggtttttttcagacagagtttttcgattagatcttacgaaataggcgctgatcgaacaacctcaatagagacgctgatgaacatgttccaggaaacgtctttgaatcattgtaagagtaacggtcttctcaatgacggctttggtcgtactcctgagatgtgtaagaagggcctcatttgggtggttacgaaaatgcaggtcgaggtgaatcgctatcctatttggggtgattctatcgaagtcaatacttgggtctccgagtcggggaaaaacggtatgggtcgtgattggctgataagtgattgcagtacaggagaaattcttgtaagagcaacgagcgtgtgggctatgatgaatcaaaagacgagaagattgtcaaaatttccatttgaggttcgacaagagatagcgcctaattttgtcgactctgttcctgtcattgaagacgatcgaaaattacacaagcttgatgtgaagacgggtgattccattcacaatggtctaactccaaggtggaatgacttggatgtcaatcagcacgttaacaatgtgaaatacattgggtggattctcaagagtgttccaacagatgtttttgaggcccaggagctatgtggagtcacccttgagtataggcgggaatgcggaagggacagtgtgatggagtccgttacagctatggatccctcaaaagagggagaccgatcagtgtaccagcaccttcttcggcttgaggatggggctgatatcgccataggcagaaccgagtggcggccgaagaatgcaggagccaatggggcaatatcaacaggaaagacttcaaatagaaactctgtctct

SEQ ID NO: 20 - protein, 394 aa, Cuphea hookeriana
MVAAAASSAFFSVPTPGISPKPGKFGNGGFQVKANANAHPSLKSGSLETEDDTSSSSPPPRTFINQLPDWSMLLSAITTIFGAAEKQWMMLDRKSKRPDMLMEPFGVDSIVQDGVFFRQSFSIRSYEIGADRTTSIETLMNMFQETSLNHCKSNGLLNDGFGRTPEMCKKGLIWVVTKMQVEVNRYPIWGDSIEVNTWVSESGKNGMGRDWLISDCSTGEILVRATSVWAMMNQKTRRLSKFPFEVRQEIAPNFVDSVPVIEDDRKLHKLDVKTGDSIHNGLTPRWNDLDVNQHVNNVKYIGWILKSVPTDVFEAQELCGVTLEYRRECGRDSVMESVTAMDPSKEGDRSVYQHLLRLEDGADIAIGRTEWRPKNAGANGAISTGKTSNRNSVS

SEQ ID NO: 21 - DNA, 1128 bp, Cuphea hookeriana
  CDS (1)..(1128)
atgttgaagctttcttgcaatgccgccaccgaccagattctgtcgtcggccgtggctcaaaccgcattatggggtcaacccagaaacagatccttttcaatgtccgcccggagaaggggagccgtttgctgcgcgcctccagctgctggaaagccccctgccatgaccgctgttatcccaaaagacggggtggcctcgtccgggtccggcagcctggccgaccagctgaggctcgggagccgtacgcagaatgggctgtcgtacacggagaagttcattgtcaggtgctacgaggtcggtattaacaagacagccactgtcgaaaccatggccaatctcttgcaggaagtaggttgtaaccatgctcagagtgttggattctcaactgacgggtttgcgacgacgcctaccatgaggaaattgaatctgatatgggttactgctcgaatgcacatagaaatttataagtacccagcatggagtgatgtggttgaaatcgagacttggtgccaaagtgaaggaagaatcggaacaagaagggattggattctcaaggattatggtaatggtgaagttattggaagagccacaagcaagtgggtgatgatgaaccagaacactagacgactccaaaaagttgatgattccgttcgagaagagtatatggttttctgtccacgcgaaccaaggttatcatttcctgaagagaacaatcggagtttgagaaaaatatctaaattggaagatcctgctgagtattcgagacttggtcttacgcctagaagagctgatctggatatgaaccagcatgtcaacaacgttgcttacataggttgggctctggagagtgtacctcaagaaataatcgactcttatgagctggaaactatcactctggactacagaagagaatgccaacaggatgacgtagtcgattcgctcaccagtgttctgtcagatgaggaatcaggaacattaccagagctcaagggaacaaatggatctgcatccaccccactgaaacgtgaccatgatggctctcgccagttcttgcacttgctgaggctctcccccgacgggctagaaataaaccgtggccgaactgaatggagaaagaaatccacgaaa

SEQ ID NO: 22 - protein, 376 aa, Cuphea hookeriana
MLKLSCNAATDQILSSAVAQTALWGQPRNRSFSMSARRRGAVCCAPPAAGKPPAMTAVIPKDGVASSGSGSLADQLRLGSRTQNGLSYTEKFIVRCYEVGINKTATVETMANLLQEVGCNHAQSVGFSTDGFATTPTMRKLNLIWVTARMHIEIYKYPAWSDVVEIETWCQSEGRIGTRRDWILKDYGNGEVIGRATSKWVMMNQNTRRLQKVDDSVREEYMVFCPREPRLSFPEENNRSLRKISKLEDPAEYSRLGLTPRRADLDMNQHVNNVAYIGWALESVPQEIIDSYELETITLDYRRECQQDDVVDSLTSVLSDEESGTLPELKGTNGSASTPLKRDHDGSRQFLHLLRLSPDGLEINRGRTEWRKKSTK

SEQ ID NO: 23 - DNA, 39 bp, Cuphea hookeriana
ctactactactagcatgcatgcttgattggaaacctaag

SEQ ID NO: 24 - DNA, 32 bp, Cuphea hookeriana
gcggccgcggtaccatgcttgataggaaatct

SEQ ID NO: 25 - DNA, 36 bp, Cuphea hookeriana
ctactactactagcatgcatgaccgctgttatccca

SEQ ID NO: 26 - DNA, 36 bp, Cuphea hookeriana
catcatcatcatggtaccgaccagggctcccttcta

SEQ ID NO: 27 - DNA, 32 bp, Cuphea hookeriana
gcggccgcctgcagaaagctcccgagcccctt

SEQ ID NO: 28 - DNA, 36 bp, Cuphea hookeriana
catcatcatcatggtaccatgtaagagactcctcta

SEQ ID NO: 29 - DNA, 36 bp, Cuphea hookeriana
gaattctggccagacatgcatgatcggaaatccaag

SEQ ID NO: 30 - DNA, 63 bp, Cuphea hookeriana
gaattctctagagtaccagatctctaagagaccgagtttccatttgaagtctttcccgttgat

(19) United States
(12) Patent Application Publication
(10) Pub. No.: US 2002/0178467 A1
(43) Pub. Date: Nov. 28, 2002

(54) Plastid transit peptide sequences for efficient plastid targeting

(76) Inventor: Katayoon Dehesh, Vacaville, CA (US)

Correspondence Address: MONSANTO COMPANY, 800 N. LINDBERGH BLVD., ATTENTION: G.P. WUELLNER, IP PARALEGAL, (E2NA), ST. LOUIS, MO, 63167, US

(21) Appl. No.: 09/854,286

(22) Filed: May 11, 2001

Related U.S. Application Data
(60) Provisional application No. 60/203,618, filed on May 12, 2000.

(51) Int. Cl. A01H 5/00; C07H 21/04; C12N 15/82

(52) U.S. Cl. 800/279; 800/281; 435/320.1; 536/23.2

(57) ABSTRACT

Novel nucleic acid sequences encoding plastid transit peptides are provided. The plastid transit peptide is capable of translocating a protein operably linked to the transit peptide, to a plant cell plastid. Also considered are arnino acid and nucleic acid sequences of the plastid transit peptides of the invention and the use of such sequences to produce DNA constructs. Provided are methods for the increased translocation of proteins to a plant cell plastid using the nucleic acid sequences of the present invention.

This application claims the benefit of U.S. Provisional Application Ser. No. 60/203,618, filed May 12, 2000.

### TECHNICAL FIELD

The present invention is directed to the field of plant genetic engineering. More particularly to nucleic acid molecules and DNA constructs encoding plastid transit peptides and amino acid compositions thereof, and methods related thereto.

### BACKGROUND OF THE INVENTION

Plant cells contain distinct subcellular compartments delimited by membranes composed of membrane lipids. In photosynthetic leaf plants, the most conspicuous organelles are the chloroplasts. The chloroplast present in leaf cells is one developmental stage of this organelle. Proplastids, etioplasts, amyloplasts, and chromoplasts represent different stages. The majority of chloroplast proteins are coded by nuclear genes synthesized in the cytoplasm and then imported into the chloroplast. Import is associated with the removal of an amino terminal portion of the protein, referred to as the chloroplast transit peptide or the transit peptide. The transit peptide is linked to the mature peptide by an amino acid sequence, normally requiring at least two amino acids, which is recognized by a specific protease associated with the chloroplast. Thus, the proform of the mature peptide is translocated to the chloroplast and processed as a result of recognition by one or more proteins.

In all plants studied, fatty acid biosynthesis occurs predominantly in the stroma of plastids. This requires that the majority of the enzymes involved in fatty acid biosynthesis must be transported from the cytosol into the plastid. Most plastid proteins are encoded by nuclear genes and are synthesized as higher molecular weight precursors that include an amino-terminal extension referred to as the transit peptide. The transit peptide is an essential sequence for transporting proteins into plastids and can be used in trans to direct foreign proteins into chloroplasts (Mishkind et al., 1985; Chua and Schmidt, 1978; Lubben et al.,1988 ). Despite many studies over the years, the structural features that make one transit peptide quantitatively better than another is still not well understood (von Heijne et al., 1989; von Heijne and Nishikawa, 1991).

For many purposes in the manipulation and transformation of plant cells to provide particular functions in the plant cell, it will be desirable that the gene that is introduced into the plant cell results in a product that is translocated to the plastid and functions in the plastid. The identification of efficient transit peptides is needed in the art.

### SUMMARY OF THE INVENTION

The present invention is directed to DNA constructs comprising chimeric plastid transit peptide coding sequences fused to agronomic genes of interest, and in particular, the plastid transit peptide coding sequences that enhance transport of chimeric proteins into seed plastids. The plastid transit polypeptides and polynucleotides of the present invention comprise those derived from Cuphea acyl-ACP thioesterases. More particularly, to transit polypeptide molecules comprising SEQ ID NO:2, SEQ ID NO:4, SEQ ID NO:6, SEQ ID NO:8, SEQ ID NO: 10, SEQ ID NO: 12, and SEQ ID NO: 14.

In yet another aspect of the present invention, DNA constructs comprising chimeric DNA molecules encoding plastid transit peptides of Cuphea acyl-ACP thioesterase sequences of the present invention are provided. More particularly to DNA molecules comprising SEQ ID NO: 1, SEQ ID NO:3, SEQ ID NO:5, SEQ ID NO:7, SEQ ID NO:9, SEQ ID NO:11, SEQ ID NO: 13.

In one aspect of the present invention, a method is provided to enhance translocation of a chimeric polypeptide to plant cell plastids comprising constructing a DNA construct encoding a chimeric fusion protein having a plastid transit peptide selected from the group consisting of SEQ ID NO:2, SEQ ID NO:4, SEQ ID NO:6, SEQ ID NO:8, SEQ ID NO:10, SEQ ID NO:12, and SEQ ID NO: 14; and expressing the chimeric fusion protein in a transgenic plant cell. More preferably, a DNA construct encoding a chimeric fusion protein of SEQ ID NO:2 and a coding sequence of interest transformed into a plant cell. The recombinant plant cells that contain such constructs are also part of the present invention.

The modified plants, seeds and oils obtained by the expression of proteins using the sequences, constructs and methods of the present invention are also considered part of the invention.

### BRIEF DESCRIPTION OF THE DRAWINGS

FIG. 1. Comparison of transit peptide efficiency in a Pea chloroplast import assay.

FIG. 2. Plasmid map of pCGN9346

FIG. 3. Plasmid map of pCGN9347

### DETAILED DESCRIPTION OF THE INVENTION

In accordance with the subject invention, nucleic acid sequences are provided that are capable of encoding sequences of amino acids, such as, a protein, a peptide or a polypeptide, that comprise plastid transit peptides of Cuphea acyl-ACP thioesterase (also referred to herein as PTP). The novel DNA constructs comprising the nucleic acid sequences find use in the preparation of constructs to direct their expression in a host cell and transport a chimeric protein into a plastid. In addition, nucleic acid sequences encoding acyl-ACP thioesterases are also provided. Such sequences also find use in the preparation of DNA constructs containing the PTPs of the present invention to direct chimeric acyl-ACP thioesterase proteins into a plant cell plastid. Furthermore, the novel DNA constructs find use in modifying the fatty acid composition of a plant cell.

### Isolated Polynucleotides and Polypeptides

A first aspect of the present invention relates to isolated plastid transit peptide (also referred to herein as PTP) polynucleotides, wherein these PTP encoding polynucleotides are fused to an agronomic gene of interest to direct transport to plant cell plastids. Of particular interest are the isolated polynucleotides encoding plastid transit peptides obtained from Cuphea acyl-ACP thioesterase sequences. The polynucleotide sequences of the present invention encode the polypeptides having a deduced amino acid sequence of SEQ ID NO:2 (C. palustris FatB2), SEQ ID NO:4 (C. palustris FatB 1) , SEQ ID NO:6 (C. hookeriana FatA1), SEQ ID NO:8 (C. hookeriana FatB1), SEQ ID NO: 10 (C. hookeriana FatB1-1), SEQ ID NO: 12 (C. hookeriana FatB2), and SEQ ID NO: 14 (C. hookeriana FatB3). The present invention also provides chimeric polynucleotides that encode the PTPs and encode acyl-ACP thioesterases, and in particular acyl-ACP thioesterase sequences obtainable from Cuphea species.

The invention also provides the chimeric coding sequence for the mature polypeptide or a fragment thereof in a reading frame with other coding sequences, such as those encoding a leader or secretory sequence, a pre-, pro-, or prepro- protein sequence. The polynucleotide can also include non-coding sequences, including for example, but not limited to, non-coding 5′ and 3′ sequences, such as the transcribed, untranslated sequences, termination signals, ribosome binding sites, sequences that stabilize MRNA, introns, polyadenylation signals, and additional coding sequence that encodes additional amino acids. For example, a marker sequence can be included to facilitate the purification of the fused polypeptide. Polynucleotides of the present invention also include polynucleotides comprising a structural gene and the naturally associated sequences that control gene expression.

The invention also includes polynucleotides of the formula:

X-(R₁)ₙ-(R₂)-(R₃)ₙY

wherein, at the 5′ end, X is hydrogen, and at the 3′ end, Y is hydrogen or a metal, R₁ and R₃ are any nucleic acid residue, n is an integer between 1 and 3000, preferably between 1 and 1000 and R₂ is a nucleic acid sequence of the invention, particularly a nucleic acid sequence selected from the group set forth in the Sequence Listing and preferably SEQ ID NOs: 1, 3, 5, 7, 9, 11, and 13.

In the formula, R₂ is oriented so that its 5′ end residue is at the left, bound to R₁, and its 3′ end residue is at the right, bound to R₃. Any stretch of nucleic acid residues denoted by either R group, where R is greater than 1, may be either a heteropolymer or a homopolymer, preferably a heteropolymer.

The invention also relates to variants of the polynucleotides described herein that encode for variants of the polypeptides of the invention. Variants that are fragments of the polynucleotides of the invention can be used to synthesize full-length polynucleotides of the invention. Preferred embodiments are polynucleotides encoding polypeptide variants wherein 5 to 10, 1 to 5, 1 to 3, 2, 1 or no amino acid residues of a polypeptide sequence of the invention are substituted, added or deleted, in any combination. Particularly preferred are substitutions, additions, and deletions that are silent such that they do not alter the properties or activities of the polynucleotide or polypeptide.

Nucleotide sequences encoding the Cuphea acyl-ACP thioesterases and the PTPs of the present invention may be obtained from natural sources or be partially or wholly artificially synthesized. They may directly correspond to a PTP endogenous to a natural source or contain modified amino acid sequences, such as sequences that have been mutated, truncated, increased or the like. Plastid transit peptides may be obtained by a variety of methods, including but not limited to, partial or homogenous purification of protein extracts, protein modeling, nucleic acid probes, antibody preparations and sequence comparisons. Typically a PTP will be derived in whole or in part from a natural source. A natural source includes, but is not limited to, eukaryotic sources, including, yeasts, plants, including algae, and the like.

Of special interest are PTP that are obtainable from plant acyl-ACP thioesterase sources, including those that are obtained, from Cuphea, or from additional sources that are obtainable through the use of these sequences. “Obtainable” refers to those PTPs that have sufficiently similar sequences to that of the sequences provided herein to provide a biologically active polypeptide of the present invention.

Further preferred embodiments of the invention that are at least 50%, 60%, or 70% identical over their entire length to a polynucleotide encoding a polypeptide of the invention, and polynucleotides that are complementary to such polynucleotides. More preferable are polynucleotides that comprise a region that is at least 80% identical over its entire length to a polynucleotide encoding a polypeptide of the invention and polynucleotides that are complementary thereto. In this regard, polynucleotides at least 90% identical over their entire length are particularly preferred, those at least 95% identical are especially preferred. Further, those with at least 97% identity are highly preferred and those with at least 98% and 99% identity are particularly highly preferred, with those at least 99% being the most highly preferred.

Preferred embodiments are polynucleotides that encode polypeptides that retain substantially the same biological function or activity as the mature polypeptides encoded by the polynucleotides set forth in the Sequence Listing.

The invention further relates to polynucleotides that hybridize to the above-described sequences. In particular, the invention relates to polynucleotides that hybridize under stringent conditions to the above-described polynucleotides. As used herein, the terms “stringent conditions” and “stringent hybridization conditions” mean that hybridization will generally occur if there is at least 95% and preferably at least 97% identity between the sequences. An example of stringent hybridization conditions is overnight incubation at 42° C. in a solution comprising 50% formamide, 5x SSC (150 mM NaCl, 15 mM trisodium citrate), 50 mM sodium phosphate (pH 7.6), 5x Denhardt's solution, 10% dextran sulfate, and 20 micrograms/milliliter denatured, sheared salmon sperm DNA, followed by washing the hybridization support in 0.1x SSC at approximately 65° C. Other hybridization and wash conditions are well known and are exemplified in Sambrook, et al., Molecular Cloning: A Laboratory Manual, Second Edition, cold Spring Harbor, N.Y. (1989), particularly Chapter 11.

The invention also provides a polynucleotide consisting essentially of a polynucleotide sequence obtainable by screening an appropriate library containing the complete gene for a polynucleotide sequence set for in the Sequence Listing under stringent hybridization conditions with a probe having the sequence of said polynucleotide sequence or a fragment thereof; and isolating said polynucleotide sequence. Fragments useful for obtaining such a polynucleotide include, for example, probes and primers as described herein.

As discussed herein regarding polynucleotide assays of the invention, for example, polynucleotides of the invention can be used as a hybridization probe for RNA, cDNA, or genomic DNA to isolate full length cDNAs or genomic clones encoding a polypeptide and to isolate cDNA or genomic clones of other genes that have a high sequence similarity to a polynucleotide set forth in the Sequence Listing. Such probes will generally comprise about 11 homologous nucleotide bases. Preferably such probes will have about 20 nucleotide bases, more preferably about 30 nucleotide bases and can comprise about 50 bases. Particularly preferred probes will have between 30 bases and 50 bases, inclusive.

The coding region of each gene that comprises or is comprised by a polynucleotide sequence set forth in the Sequence Listing may be isolated by screening using a DNA sequence provided in the Sequence Listing to synthesize an oligonucleotide probe. A labeled oligonucleotide having a sequence complementary to that of a gene of the invention is then used to screen a library of cDNA, genomic DNA or mRNA to identify members of the library that hybridize to the probe. For example, synthetic oligonucleotides are prepared that correspond to the N-terminal sequence of the polypeptide. The partial sequences so prepared can then be used as probes to obtain thioesterase (TE) clones from a gene library prepared from a cell source of interest. Alternatively, where oligonucleotides of low degeneracy can be prepared from particular peptides, such probes may be used directly to screen gene libraries for gene sequences.

In particular, screening of cDNA libraries in phage vectors is useful in such methods due to lower levels of background hybridization.

Typically, a sequence obtainable from the use of nucleic acid probes will show 60-70% sequence identity between the target PTP sequence and the encoding sequence used as a probe. However, lengthy sequences with as little as 50-60% sequence identity may also be obtained. The nucleic acid probes may be a lengthy fragment of the nucleic acid sequence, or may also be a shorter, oligonucleotide probe. When longer nucleic acid fragments are employed as probes (greater than about 100 bp), one may screen at lower stringencies in order to obtain sequences from the target sample that have 20-50% deviation (i.e., 50-80% sequence homology) from the sequences used as probe. Oligonucleotide probes can be considerably shorter than the entire nucleic acid sequence encoding an thioesterase enzyme, but should be about 10 nucleotide bases, preferably about 15, and more preferably about 20 nucleotide bases. A higher degree of sequence identity is desired when shorter regions are used as opposed to longer regions. It may thus be desirable to identify regions of highly conserved amino acid sequence to design oligonucleotide probes for detecting and recovering other related genes. Shorter probes are often particularly useful for polymerase chain reactions (PCR), especially when highly conserved sequences can be identified (Gould, et al., Proc. Natl. Acad. Sci. USA (1989) 86:1934-1938).

The skilled artisan will appreciate that, in many cases, an isolated cDNA sequence will be incomplete, in that the region coding for the polypeptide is truncated with respect to the 5′ terminus of the cDNA. This is a consequence of the reverse transcriptase, an enzyme with low ‘processivity’ (a measure of the ability of the enzyme to remain attached to the template during the polymerization reaction) employed during the first strand cDNA synthesis.

There are several methods available and are well know to the skilled artisan to obtain full-length cDNAs, or extend short cDNAs, for example those based on the method of Rapid Amplification of cDNA Ends (RACE) (for example, Frohman et al. (1988) Proc. Natl. Acad. Sci. USA 85:8998-9002). Recent modifications of the technique, exemplified by the Marathon™ technology (Clonetech Laboratories, Inc., Palo Alto, Calif.) for example, have significantly simplified obtaining full-length cDNA sequences.

Another aspect of the present invention relates to isolated plastid transit polypeptides. Such polypeptides include isolated polypeptides set forth in the Sequence Listing, as well as polypeptides and fragments thereof, particularly those polypeptides that exhibit thioesterase activity and also those polypeptides that have at least 50%, 60% or 70% identity, preferably at least 80% identity, more preferably at least 90% identity, and most preferably at least 95% identity to a polypeptide sequence selected from the group of sequences set forth in the Sequence Listing, and also include portions of such polypeptides, wherein such portion of the polypeptide preferably includes at least 30 amino acids and more preferably includes at least 50 amino acids. “Identity”, as is well understood in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. “Identity” can be readily calculated by known methods including, but not limited to, those described in Computational Molecular Biology, Lesk, A. M., ed., Oxford University Press, New York (1988); Biocomputing: Informatics and Genome Projects, Smith, D. W., ed., Academic Press, New York, 1993; Computer Analysis of Sequence Data, Part I, Griffin, A. M. and Griffin, H. G., eds., Humana Press, New Jersey (1994); Sequence Analysis in Molecular Biology, von Heinje, G., Academic Press (1987); Sequence Analysis Primer, Gribskov, M. and Devereux, J., eds., Stockton Press, New York (1991); and Carillo, H., and Lipman, D., SIAM J Applied Math, 48:1073 (1988). Methods to determine identity are designed to give the largest match between the sequences tested. Moreover, methods to determine identity are codified in publicly available programs. Computer programs that can be used to determine identity between two sequences include, but are not limited to, GCG (Devereux, J., et al., Nucleic Acids Research 12(1):387 (1984); suite of five BLAST programs, three designed for nucleotide sequences queries (BLASTN, BLASTX, and TBLASTX) and two designed for protein sequence queries (BLASTP and TBLASTN) (Coulson, Trends in Biotechnology, 12: 76-80 (1994); Birren, et al., Genome Analysis, 1: 543-559 (1997)). The BLAST X program is publicly available from NCBI and other sources (BLAST Manual, Altschul, S., et al., NCBI NLM NIH, Bethesda, Md. 20894; Altschul, S., et al., J. Mol. Biol., 215:403-410 (1990)). The well known Smith Waterman algorithm can also be used to determine identity.

Parameters for polypeptide sequence comparison typically include the following: Algorithm: Needleman and Wunsch, J. Mol. Biol. 48:443-453 (1970) Comparison matrix: BLOSSUM62 from Hentikoff and Hentikoff, Proc. Natl. Acad. Sci USA 89:10915-10919 (1992)

Gap Penalty: 12

Gap Length Penalty: 4

A program that can be used with these parameters is publicly available as the “gap” program from Genetics Computer Group, Madison Wisconsin. The above parameters along with no penalty for end gap are the default parameters for peptide comparisons.

Parameters for polynucleotide sequence comparison include the following:

Algorithm: Needleman and Wunsch, J. Mol. Biol. 48:443-453 (1970)

Comparison matrix: matches=+10; mismatches=0

Gap Penalty: 50

Gap Length Penalty: 3

A program that can be used with these parameters is publicly available as the “gap” program from Genetics Computer Group, Madison Wis. The above parameters are the default parameters for nucleic acid comparisons.

The invention also includes polypeptides of the formula:

X(R₁)ₙ-(R₂)-(R₃)ₙ-Y

wherein, at the amino terminus, X is hydrogen, and at the carboxyl terminus, Y is hydrogen or a metal, R₁ and R₃ are any amino acid residue, n is an integer between 1 and 1000, and R₂ is an amino acid sequence of the invention, particularly an amino acid sequence selected from the group set forth in the Sequence Listing and preferably SEQ ID NOs: 2, 4, 6, 8, 10, 12, and 14. In the formula, R₂ is oriented so that its amino terminal residue is at the left, bound to R₁, and its carboxy terminal residue is at the right, bound to R₃. Any stretch of amino acid residues denoted by either R group, where R is greater than 1, may be either a heteropolymer or a homopolymer, preferably a heteropolymer.

Polypeptides of the present invention include isolated polypeptides encoded by a polynucleotide comprising a polypeptide sequence selected from the group of a sequence contained in SEQ ID NOs: 2, 4, 6, 8, 10, 12, and 14, and fragments thereof.

Polypeptides of the present invention have been shown to have plastid translocation activity and are of interest because many proteins expressed from introduced nucleic acid constructs need to be localized to the plant cell plastid and processed for proper activity.

Fragments and variants of the polypeptides are also considered to be a part of the invention. A fragment is a variant polypeptide that has an amino acid sequence that is entirely the same as part but not all of the amino acid sequence of the previously described polypeptides. The fragments can be “free-standing” or comprised within a larger polypeptide of that the fragment forms a part or a region, most preferably as a single continuous region. Preferred fragments are biologically active fragments that are those fragments that mediate activities of the polypeptides of the invention, including those with similar activity or improved activity or with a decreased activity. Also included are those fragments that antigenic or immunogenic in an animal, particularly a human.

Variants of the polypeptide also include polypeptides that vary from the sequences set forth in the Sequence Listing by conservative amino acid substitutions, substitution of a residue by another with like characteristics. In general, such substitutions are among Ala, Val, Leu and Ile; between Ser and Thr; between Asp and Glu; between Asn and Gln; between Lys and Arg; or between Phe and Tyr. Particularly preferred are variants in which 5 to 10; 1 to 5; 1 to 3 or one amino acid(s) are substituted, deleted, or added, in any combination.

Variants that are fragments of the polypeptides of the invention can be used to produce the corresponding full length polypeptide by peptide synthesis. Therefore, these variants can be used as intermediates for producing the full-length polypeptides of the invention.

The polynucleotides and polypeptides of the invention can be used, for example, in the transformation of various host cells, as further discussed herein.

The invention also provides polynucleotides that encode a polypeptide that is a mature protein plus additional amino or carboxyl-terminal amino acids, or amino acids within the mature polypeptide (for example, when the mature form of the protein has more than one polypeptide chain). Such sequences can, for example, play a role in the processing of a protein from a precursor to a mature form, allow protein transport, shorten or lengthen protein half-life, or facilitate manipulation of the protein in assays or production. It is contemplated that cellular enzymes can be used to remove any additional amino acids from the mature protein.

A precursor protein, having the mature form of the polypeptide fused to one or more prosequences may be an inactive form of the polypeptide. The inactive precursors generally are activated when the prosequences are removed. Some or all of the prosequences may be removed prior to activation. Such precursor protein are generally called proproteins.

The polynucleotide and polypeptide sequences can also be used to identify additional sequences that are homologous to the sequences of the present invention. The most preferable and convenient method is to store the sequence in a computer readable medium, for example, floppy disk, CD ROM, hard disk drives, external disk drives and DVD, and then to use the stored sequence to search a sequence database with well known searching tools. Examples of public databases include the DNA Database of Japan (DDBJ)(http:H/www.ddbj.nig.acjpl); Genbank (http:H/www.ncbi.nlm.nih.gov/web/Genbank/Index.htlm); and the European Molecular Biology Laboratory Nucleic Acid Sequence Database (EMBL)

(http://www.ebi.ac.uk/ebi docs/embl db.html). A number of different search algorithms are available to the skilled artisan, one example of which are the suite of programs referred to as BLAST programs. There are five implementations of BLAST, three designed for nucleotide sequences queries (BLASTN, BLASTX, and TBLASTX) and two designed for protein sequence queries (BLASTP and TBLASTN) (Coulson, Trends in Biotechnology, 12: 76-80 (1994); Birren, et al., Genome Analysis, 1: 543-559 (1997). Additional programs are available in the art for the analysis of identified sequences, such as sequence alignment programs, programs for the identification of more distantly related sequences, and the like, and are well known to the skilled artisan.

### Plant Constructs and Methods of Use

Of interest in the present invention, is the use of the nucleotide sequences, or polynucleotides, in recombinant DNA constructs to direct the transcription and translation of the nucleic acid sequences of interest in a host cell. Of particular interest is the use of the plastid targeting regions of the thioesterase (TE) sequences of the present invention in recombinant DNA constructs operably linked to nucleic acid sequences encoding proteins of interest to direct the expressed protein to the plant cell chloroplast and seed plastids.

As used herein, “recombinant” includes reference to a cell or vector, that has been modified by the introduction of a heterologous nucleic acid sequence or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention.

A first nucleic acid sequence is “operably linked” with a second nucleic acid sequence when the sequences are so arranged that the first nucleic acid sequence affects the function of the second nucleic-acid sequence. Preferably, the two sequences are part of a single contiguous nucleic acid molecule and more preferably are adjacent. For example, a promoter is operably linked to a gene if the promoter regulates or mediates transcription of the gene in a cell.

Of particular interest is the use of the nucleotide sequences, or polynucleotides, in recombinant DNA constructs to direct the transcription and translation (expression) of nucleic acid sequences of interest in a host cell. The expression constructs generally comprise a promoter functional in a host cell operably linked to a nucleic acid sequence encoding a gene of interest fused to a plastid transit peptide of the present invention and a transcriptional termination region functional in a host cell.

By “host cell” is meant a cell that contains a vector and supports the replication, and/or transcription or transcription and translation (expression) of the expression construct. Host cells for use in the present invention can be prokaryotic cells, such as E. coli, or eukaryotic cells such as yeast, plant, insect, amphibian, or mammalian cells. Preferably, host cells are monocotyledenous or dicotyledenous plant cells.

Of particular interest in the present invention is the use of the polynucleotides of the present invention for the preparation of constructs to direct the transcription or transcription and translation of the nucleotide sequences encoding gene name in a host plant cell. Plant expression constructs generally comprise a promoter functional in a plant host cell operably linked to a nucleic acid sequence of the present and a transcriptional termination region functional in a host plant cell.

Those skilled in the art will recognize that there are a number of constitutive and tissue specific promoters that are functional in plant cells, and have been described in the literature.

Chloroplast and plastid specific promoters, chloroplast or plastid functional promoters, and chloroplast or plastid operable promoters are also envisioned.

Constitutive promoters such as the CaMV35S or FMV35S promoters that yield high levels of expression in most plant organs. Enhanced or duplicated versions of the CaMV35S and FMV35S promoters are useful in the practice of this invention (Odell, et al. (1985) Nature 313:810-812; Rogers, U.S. Pat. No. 5,378, 619). In addition, it may also be preferred to bring about expression of the protein of interest in specific tissues of the plant, such as leaf, stem, root, tuber, seed endosperm, seed embryos, fruit, etc., and the promoter chosen should have the desired tissue and developmental specificity.

Of particular interest is the expression of the nucleic acid sequences of the present invention from transcription initiation regions that are preferentially expressed in a plant seed tissue. Examples of such seed preferential transcription initiation sequences include those sequences derived from sequences encoding plant storage protein genes or from genes involved in fatty acid biosynthesis in oilseeds. Examples of such promoters include the 5′ regulatory regions from such genes as napin (Kridl et al., Seed Sci. Res. 1:209:219 (1991), phaseolin, zein, soybean trypsin inhibitor, ACP, stearoyl-ACP desaturase, soybean α′ subunit of β-conglycinin, soy 7s, (Chen et al., Proc. Natl. Acad. Sci., 83:8560-8564 (1986) and oleosin.

Of particular interest in the present invention is the use of the polynucleotides encoding PTP to direct the localization of proteins of interest to the plant cell chloroplast or other plastidic compartment. For example, where the genes of interest for use in the methods of the present invention will be targeted to plastids, such as chloroplasts and seed plastids, the constructs will also employ the use of sequences of the present invention to direct the protein product of the gene to the plastid. Such sequences are referred to herein as chloroplast transit peptides (CTP) or plastid transit peptides (PTP). In this manner, where the gene of interest is not directly inserted into the plastid, the expression construct will additionally contain a gene encoding a transit peptide to direct the gene of interest to the plastid. The chloroplast transit peptides may be derived from the gene of interest, or may be derived from a heterologous sequence having a PTP.

Additionally, the PTP sequences of the present invention can be combined with other PTP sequences. Such transit peptides are known in the art. See, for example, Von Heijne et al. (1991) Plant Mol. Biol. Rep. 9:104-126; Clark et al. (1989) J. Biol. Chem. 264:17544-17550; della-Cioppa et al. (1987) Plant Physiol. 84:965-968; Romer et al. (1993) Biochem. Biophys. Res Commun. 196:1414-1421; and, Shah et al. (1986) Science 233:478-481. Additional transit peptides for the translocation of the protein to the endoplasmic reticulum (ER) (Chrispeels, K., (1991) Ann. Rev. Plant Phys. 42:21-53), nuclear localization signals (Raikhel, N. (1992) Plant Phys. 100:1627-1632), or vacuole may also find use in the constructs of the present invention.

Depending upon the intended use, the constructs may contain the nucleic acid sequence that encodes the entire gene of interest, or a portion thereof. Furthermore, where gene sequences used in constructs are intended for use as probes, it may be advantageous to prepare constructs containing only a particular portion of a gene encoding sequence, for example a sequence that is discovered to encode a highly conserved region.

Regulatory transcript termination regions may be provided in plant expression constructs of this invention as well. Transcript termination regions may be provided by the DNA sequence encoding the thioesterase or a convenient transcription termination region derived from a different gene source, for example, the transcript termination region that is naturally associated with the transcript initiation region. The skilled artisan will recognize that any convenient transcript termination region that is capable of terminating transcription in a plant cell may be employed in the constructs of the present invention.

A plant cell, tissue, organ, or plant into which the recombinant DNA constructs containing the expression constructs have been introduced is considered transformed, transfected, or transgenic. A transgenic or transformed cell or plant also includes progeny of the cell or plant and progeny produced from a breeding program employing such a transgenic plant as a parent in a cross and exhibiting an altered phenotype resulting from the presence of an introduced nucleic acid sequence.

The term “introduced” in the context of inserting a nucleic acid sequence into a cell, means “transfection”, or “transformation” or “transduction” and includes reference to the incorporation of a nucleic acid sequence into a eukaryotic or prokaryotic cell where the nucleic acid sequence may be incorporated into the genome of the cell (for example, chromosome, plasmid, plastid, or mitochondrial DNA), converted into an autonomous replicon, or transiently expressed (for example, transfected MRNA).

As used herein, the term “plant” includes reference to whole plants, plant organs (for example, leaves, stems, roots, etc.), seeds, and plant cells and progeny of same. Plant cell, as used herein includes, without limitation, seed suspension cultures, embryos, meristematic regions, callus tissue, leaves, roots, shoots, gametophytes, sporophytes, pollen, and microspores. The class of plants that can be used in the methods of the present invention is generally as broad as the class of higher plants amenable to transformation techniques, including both monocotyledenous and dicotyledenous plants. Particularly preferred plants include Acacia, alfalfa, aneth, apple, apricot, artichoke, arugula, asparagus, avocado, banana, barley, beans, beet, blackberry, blueberry, broccoli, brussels sprouts, cabbage, canola, cantaloupe, carrot, cassava, cauliflower, celery, cherry, chicory, cilantro, citrus, clementines, coffee, corn, cotton, cucumber, Douglas fir, eggplant, endive, escarole, eucalyptus, fennel, figs, garlic, gourd, grape, grapefruit, honey dew, jicama, kiwifruit, lettuce, leeks, lemon, lime, Loblolly pine, mango, melon, mushroom, nectarine, nut, oat, oil palm, oil seed rape, okra, onion, orange, an ornamental plant, papaya, parsley, pea, peach, peanut, pear, pepper, persimmon, pine, pineapple, plantain, plum, pomegranate, poplar, potato, pumpkin, quince, radiata pine, radicchio, radish, raspberry, rice, rye, safflower, sorghum, Southern pine, soybean, spinach, squash, strawberry, sugarbeet, sugarcane, sunflower, sweet potato, sweetgum, tangerine, tea, tobacco, tomato, triticale, turf, turnip, a vine, watermelon, wheat, yams, and zucchini.

As used herein, “transgenic plant” includes reference to a plant that comprises within its genome a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to include any cell, cell line, callus, tissue, plant part or plant, the genotype of which has been altered by the presence of heterologous nucleic acid including those transgenics initially so altered as well as those created by sexual crosses or asexual propagation from the initial transgenic.

Thus a plant having within its cells a heterologous polynucleotide is referred to herein as a transgenic plant. The heterologous polynucleotide can be either stably integrated into the genome, or can be extra-chromosomal. Preferably, the polynucleotide of the present invention is stably integrated into the genome such that the polynucleotide is passed on to successive generations. The polynucleotide is integrated into the genome alone or as part of a recombinant expression cassette. “Transgenic” is used herein to include any cell, cell line, callus, tissue, plant part or plant, the genotype of which has been altered by the presence of heterologous nucleic acids including those transgenics initially so altered as well as those created by sexual crosses or asexual reproduction of the initial transgenics.

As used herein, “heterologous” in reference to a nucleic acid is a nucleic acid that originates from a foreign species, or, if from the same species, is substantially modified from its native form in composition and/or genomic locus by deliberate human intervention. For example, a promoter operably linked to a heterologous structural gene is from a species different from that from which the structural gene was derived, or, if from the same species, one or both are substantially modified from their original form. A heterologous protein may originate from a foreign species, or, if from the same species, is substantially modified from its original form by deliberate human intervention.

As used herein, a “recombinant expression cassette” is a nucleic acid construct, generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter.

It is contemplated that the gene sequences may be synthesized, either completely or in part, especially where it is desirable to provide plant-preferred sequences. Thus, all or a portion of the desired structural gene (that portion of the gene that encodes the protein) may be synthesized using codons preferred by a selected host. Host-preferred codons may be determined, for example, from the codons used most frequently in the proteins expressed in a desired host species.

One skilled in the art will readily recognize that antibody preparations, nucleic acid probes (DNA and RNA) and the like may be prepared and used to screen and recover “homologous” or “related” thioesterase from a variety of plant sources. Homologous sequences are found when there is an identity of sequence, that may be determined upon comparison of sequence information, nucleic acid or amino acid, or through hybridization reactions between a known TE and a candidate source. Conservative changes, such as Glu/Asp, Val/Ile, Ser/Thr, Arg/Lys and Gln/Asn may also be considered in determining sequence homology. Amino acid sequences are considered homologous by as little as 25% sequence identity between the two complete mature proteins. (See generally, Doolittle, R. F., OF URFS and ORFS (University Science Books, CA, 1986.)

Thus, other plastid transit peptides can be obtained from the specific exemplified sequences provided herein. Furthermore, it will be apparent that one can obtain natural and synthetic PTPs, including modified amino acid sequences and starting materials for synthetic-protein modeling from the exemplified PTPs and from PTPs that are obtained through the use of such exemplified sequences. Modified amino acid sequences include sequences that have been mutated, truncated, increased and the like, whether such sequences were partially or wholly synthesized. Sequences that are actually purified from plant preparations or are identical or encode identical proteins thereto, regardless of the method used to obtain the protein or sequence, are equally considered naturally derived.

For immunological screening, antibodies to the TE protein can be prepared by injecting rabbits or mice with the purified protein or portion thereof, such methods of preparing antibodies being well known to those in the art. Either monoclonal or polyclonal antibodies can be produced, although typically polyclonal antibodies are more useful for gene isolation. Western analysis may be conducted to determine that a related protein is present in a crude extract of the desired plant species, as determined by cross-reaction with the antibodies to the TE protein.

When cross-reactivity is observed, genes encoding the related proteins are isolated by screening expression libraries representing the desired plant species. Expression libraries can be constructed in a variety of commercially available vectors, including lambda gt11, as described in Sambrook, et al. (Molecular Cloning: A Laboratory Manual, Second Edition (1989) Cold Spring Harbor Laboratory, Cold Spring Harbor, N.Y.).

The nucleic acid sequences encoding the plastid transit peptides of the present invention will find many uses. For example, recombinant constructs can be prepared that can be used as probes, or that will provide for expression of the proteins of interest in host cells to direct the expressed protein of interest to the plant cell plastid.

As discussed above, nucleic acid sequence encoding a plastid transit peptide or chimeric acyl-ACP thioesterase of this invention may include genomic, cDNA or mRNA sequence. By “encoding” is meant that the sequence corresponds to a particular amino acid sequence either in a sense or anti-sense orientation. By “extrachromosomal” is meant that the sequence is outside of the plant genome of which it is naturally associated. By “recombinant” is meant that the sequence contains a genetically engineered modification through manipulation via mutagenesis, restriction enzymes, and the like.

Once the desired nucleic acid sequence is obtained, it may be manipulated in a variety of ways. Where the sequence involves non-coding flanking regions, the flanking regions may be subjected to resection, mutagenesis, etc. Thus, transitions, transversions, deletions, and insertions may be performed on the naturally occurring sequence. In addition, all or part of the sequence may be synthesized. In the structural gene, one or more codons may be modified to provide for a modified amino acid sequence, or one or more codon mutations may be introduced to provide for a convenient restriction site or other purpose involved with construction or expression. The structural gene may be further modified by employing synthetic adapters, linkers to introduce one or more convenient restriction sites, or the like.

The nucleic acid or amino acid sequences encoding a plastid transit peptide or thioesterase of this invention may be combined with other non-native, or “heterologous”, sequences in a variety of ways. By “chimeric” sequences is meant any amino acid sequence or nucleic acid sequence that is not naturally found joined to the PTP, including, for example, combinations of nucleic acid sequences from the same plant that are not naturally found joined together.

The DNA sequence encoding a PTP of a TE of this invention may be employed in conjunction with a part of the gene sequences normally associated with the sequence. In its component parts, a DNA sequence encoding a plastid transit peptide of the present invention is combined in a DNA construct having, in the 5′ to 3′ direction of transcription, a transcription initiation control region capable of promoting transcription and translation in a host cell, a DNA sequence encoding protein of interest and a transcription termination region.

Potential host cells include both prokaryotic cells, such as E. coli and eukaryotic cells such as yeast, insect, amphibian, or mammalian cells. A host cell may be unicellular or found in a multicellular differentiated or undifferentiated organism depending upon the intended use. Preferably, host cells of the present invention include plant cells, both monocotyledenous and dicotyledenous. Cells of this invention may be distinguished by having a PTP foreign to the wild-type cell present therein, for example, by having a recombinant nucleic acid construct encoding a chloroplast transit peptide of the present invention therein.

The methods used for the transformation of the host plant cell are not critical to the present invention. The transformation of the plant is preferably permanent, i.e., by integration of the introduced expression constructs into the host plant genome, so that the introduced constructs are passed onto successive plant generations. The skilled artisan will recognize that a wide variety of transformation techniques exist in the art, and new techniques are continually becoming available. Any technique that is suitable for the target host plant can be employed within the scope of the present invention. For example, the constructs can be introduced in a variety of forms including, but not limited to as a strand of DNA, in a plasmid, or in an artificial chromosome. The introduction of the constructs into the target plant cells can be accomplished by a variety of techniques, including, but not limited to calcium-phosphate-DNA co- precipitation, electroporation, microinjection, Agrobacterium infection, liposomes or microprojectile transformation. The skilled artisan can refer to the literature for details and select suitable techniques for use in the methods of the present invention.

Normally, included with the DNA construct will be a structural gene having the necessary regulatory regions for expression in a host and providing for selection of transformant cells. The gene may provide for resistance to a cytotoxic agent, e.g. antibiotic, heavy metal, toxin, etc., complementation providing prototrophy to an auxotrophic host, viral immunity or the like. Depending upon the number of different host species the expression construct or components thereof are introduced, one or more markers may be employed, where different conditions for selection are used for the different hosts.

Where Agrobacterium is used for plant cell transformation, a vector may be used that may be introduced into the Agrobacterium host for homologous recombination with T-DNA or the Ti- or Ri-plasmid present in the Agrobacterium host. The Ti- or Ri-plasmid containing the T-DNA for recombination may be armed (capable of causing gall formation) or disarmed (incapable of causing gall formation), the latter being permissible, so long as the vir genes are present in the transformed Agrobacterium host. The armed plasmid can give a mixture of normal plant cells and gall.

In some instances where Agrobacterium is used as the vehicle for transforming host plant cells, the expression or transcription construct bordered by the T-DNA border region(s) will be inserted into a broad host range vector capable of replication in E. coli and Agrobacterium, there being broad host range vectors described in the literature. Commonly used is pRK2 or derivatives thereof. See, for example, Ditta, et al., (Proc. Nat. Acad. Sci., U.S.A. (1980) 77:7347-7351) and EPA 0 120 515, that are incorporated herein by reference. Alternatively, one may insert the sequences to be expressed in plant cells into a vector containing separate replication sequences, one of which stabilizes the vector in E. coli, and the other in Agrobacterium. See, for example, McBride and Summerfelt (Plant Mol. Biol. (1990) 14:269- 276), wherein the pRiHRI (Jouanin, et al., Mol. Gen. Genet. (1985) 201:370-374) origin of replication is utilized and provides for added stability of the plant expression vectors in host Agrobacterium cells.

Included with the expression construct and the T-DNA will be one or more markers, that allow for selection of transformed Agrobacterium and transformed plant cells. A number of markers have been developed for use with plant cells, such as resistance to chloramphenicol, kanamycin, the aminoglycoside G418, hygromycin, or the like. The particular marker employed is not essential to this invention, one or another marker being preferred depending on the particular host and the manner of construction.

For transformation of plant cells using Agrobacterium, explants may be combined and incubated with the transformed Agrobacterium for sufficient time for transformation, the bacteria killed, and the plant cells cultured in an appropriate selective medium. Once callus forms, shoot formation can be encouraged by employing the appropriate plant hormones in accordance with known methods and the shoots transferred to rooting medium for regeneration of plants. The plants may then be grown to seed and the seed used to establish repetitive generations and for isolation of vegetable oils.

There are several possible ways to obtain the plant cells of this invention that contain multiple expression constructs. Any means for producing a plant comprising a construct having a nucleic acid sequence of the present invention, and at least one other construct having another DNA sequence encoding an enzyme are encompassed by the present invention. For example, the expression construct can be used to transform a plant at the same time as the second construct either by inclusion of both expression constructs in a single transformation vector or by using separate vectors, each of which express desired genes. The second construct can be introduced into a plant that has already been transformed with the first expression construct, or alternatively, transformed plants, one having the first construct and one having the second construct, can be crossed to bring the constructs together in the same plant.

Of special interest is the use of the plastid transit peptides of the present invention in plant transformation constructs to provide for targeting of proteins expressed from DNA sequences of interest to the plant cell chloroplasts and seed plastids. Such expression constructs utilizing the plastid transit peptides of the present invention provide for the plastidial targeting of genes involved in many applications of plant genetic engineering. Genes for such applications include, but are not limited to, genes for improved agronomic traits, such as herbicide tolerance, various disease resistance genes, and other stress tolerance genes. Genes involved in quality traits may also find use in the expression constructs utilizing the transit peptides of the present invention. For example, genes involved in fatty acid biosynthesis, carotenoid biosynthesis, and the like find use in the plastid transit peptide expression constructs of the present invention.

DNA sequences encoding for proteins involved in herbicide tolerance are known in the art, and include, but are not limited to DNA sequences encoding for 5-enolpyruvylshikimate-3-phosphate synthase (EPSPS, described in U.S. Pat. Nos. 5,627,061, and 5,633,435, Padgette et al. (1996) Herbicide Resistant Crops, Lewis Publishers, 53-85, and in Penaloza-Vazquez, et al. (1995) Plant Cell Reports 14:482-487) and aroA (U.S. Pat. No. 5,094,945) for glyphosate tolerance, bromoxynil nitrilase (Bxn) for Bromoxynil tolerance (U.S. Pat. No. 4,810,648), phytoene desaturase (crtI, Misawa et al, (1993) Plant Journal 4:833-840, and (1994) Plant Jour 6:481-489) for tolerance to norflurazon, acetohydroxyacid synthase (AHAS, Sathasiivan et al. (1990) Nucl. Acids Res. 18:2188-2193)) and the bar gene for tolerance to glufosinate (DeBlock, et al. (1987) EMBO J. 6:2513-2519.

DNA sequences providing tolerance to insects are known in the art, and include, but are not limited to DNA sequences encoding insecticidal proteins, for example those isolated from Bacillus thuringiensis, Xenorhabdus sp. and Photorhabdus sp.

In addition, since the plastid is the site for production of fatty acids, by introducing various proteins into the chloroplast, one may enhance the production of fatty acids or modify the unsaturated character of the fatty acids, both as to number and site. Various enzymes that may be involved in such a function include acyl carrier protein (ACP), acetyl-CoA ACP transacylase, acyl-ACP thioesterase, malonyl-CoA ACP transacylase, β-ketoacyl-ACP, synthetase, etc. Further more, DNA sequences involved in the production of carotenoids are known in the art, and include but are not limited to the genes described in PCT Patent Application WO 98/06862, the entirety of which is incorporated herein by reference.

The invention now being generally described, it will be more readily understood by reference to the following examples that are included for purposes of illustration only and are not intended to limit the present invention.

### EXAMPLES

### EXAMPLE 1

Cuphea hookerianna plants were propagated from a cutting originally obtained from USDA in Ames, Iowa. Plants were grown at 28° C. with 16 hours of light and 8 hours of dark, until flowering, at which time the dark period was increased to 18 hours and the temperature was decreased to 22° C. Pea seeds (var. Little Marvel) were obtained from Olds Seed Co. ( Madison, Wis.). Total cellular RNA was isolated from developing seed according to Jones et al. (1995) Plant Cell, 7:359-371) and was used for double-stranded cDNA synthesis. Commercial kits were used for cDNA synthesis and library construction (Uni-Zap cloning, Stratagene, La Jolla, Calif.). Approximately 500,000 unamplified recombinant phage were plated and the plaques were then transferred to nitrocellulose . Filters were treated as previously reported (Dehesh et al. (1996) Plant Physiol. 110:203-210) and hybridized with either ChFatB1 (Jones et al., (1995) Plant Cell, 7:359-371) or CtFatAl (Knutzon et al., (1992) Proc Natl Acad Sci USA 89:2624-2628) as probes. Membranes were washed under low-stringency conditions: twice for 30 minutes at room temperature in 2x wash solution (2x SSC, 5mM EDTA, 1.5 mM sodium pyrophosphate, 0.5% SDS). Two FatB type TEs were isolated and designated, ChFatBl-1 (SEQ ID NO: 17 and the deduced amino acid sequence is provided in SEQ ID NO: 18) and ChFatB3 (SEQ ID NO: 19 and the deduced amino acid sequence is provided in SEQ ID NO:20) and one FatA type TE was identified, ChFatA1 (SEQ ID NO:21 and the deduced amino acid sequence is provided in SEQ ID NO:22). Both strands of the cDNAs were sequenced completely using an automated ABI 373A sequencer (Applied Biosystems, Foster City, Calif.). DNA and polypeptide sequence analysis were performed using the programs of Macvector (DNAStar Inc., Madison, Wis.). All three clones (Ch FatB1-1, ChFatB3 and ChFatAl) are full-length, encoding predicted polypeptides of 414 and 394 and 376 amino acids, respectively, with molecular masses of 45.7 and 43.8 and 42.2 kDa and pls of 8.06, 6.12 and 8.57, respectively. An analysis generated from sequence comparison along the entire length of the encoded polypeptide of these clones, indicates that ChFatB1-1 and ChFatB1 are 91% similar, ChFatB2 and CpFatBl are 80% similar, ChFatB3 and CpFatB2 are 83% similar. ChFatA1 shows the lowest relatedness from the FatB class of enzymes with 30% similarity between them.

### EXAMPLE 2

To clone the mature portion of ChFatB1-1, ChFatB3 and ChFatA1 into the pQE30 (QlAexpress; Qiagene Inc., Chatsworth, Calif.), the following oligonucleotide primers representing the 5′ end sequence,

ChFatB1:-l CTACTACTACTAGCATGCATGCTTGATTGGAAACCTAAG (SEQ ID NO:23)

ChFatB3 GCGGCCGCGGTACCATGCTTGATAGGAAATCT(SEQ ID NO:24)

ChFatA1 CTACTACTACTAGCATGCATGACCGCTGTTATCCCA(SEQ ID NO:25)

and primers representing the 3′-end of the clones,

ChFatB1:-i CATCATCATCATGGTACCGACCAGGGCTCCCTTCTA (SEQ ID NO:26)

ChFatB3 GCGGCCGCCTGCAGAAAGCTCCCGAGCCCCTT(SEQ ID NO:27)

ChFatA1 CATCATCATCATGGTACCATGTAAGAGACTCCTCTA(SEQ ID NO:28)

are synthesized. Subsequently, standard PCR technology is utilized to amplify the mature portions of the clones. These PCR products, except for the ChFatB3 PCR product, are cloned as SphI-AspI fragments into the SphI-KpnI sites of pQE30 vectors. The ChFatB3 PCR product however is cut with Asp718 and PstI and is cloned into corresponding sites of pQE30 vector. E. coli strain DH5a is transformed with these plasmids and following the verification of the sequence the plasmids are transferred into M15 [pREP4] strains. Transformed M15 [pREP4] strains are grown at 37° C. to an OD600 of 0.7-0.8, induced with 2 mM IPTG for 1 hour and harvested. Cells are sedimented by centrifugation, resuspended in TE assay buffer (Voelker et al., (1992) Science 257:72-74), and lysed by 3×5 seconds of sonication. Debris is sedimented by a 15 min centrifugation at 14,000x g and supernatants are analyzed on SDS-polyacrylamide gels to verify expression and stored at −20° C. for enzyme activity assay. Activity assays are carried out according to Pollard et al. (1991) Arch Biochem Biophys 284:306-312). Protein measurements are performed using BCA* protein assay kit obtained from Pierce (Rockford, Ill.).

To measure the in vitro TE (thioesterase) activity of ChFatB1-1, ChFatB3 and ChFatAt in E. coli, all three cDNAs are cloned into the QlAexpress (Qiagen, Germantown, Md.) plasmid, that then allows high-level bacterial expression of recombinant protein with an N-terminal 6xHis affinity tag. The mature portions of ChFatB1-1 and ChFatB3 and ChFatA1, as defined by sequence homology with other TEs (Jones et al., (1995) Plant Cell, 7:359-371; Dehesh et al., (1996) Plant Physiol. 110:203-210; Knutzon et al., (1992) Proc Natl Acad Sci USA 89:2624-2628), are fused in-frame to the 6xHis tag expression cassettes. Crude lysates of transformed E. coli strains expressing these clones are assayed for in vitro acyl-ACP hydrolytic activity as previously reported (Pollard et al., (1991) Arch Biochem Biophys 284:306-312). The hydrolytic activity was measured one hour after induction. These results show that ChFatB1-1 encodes an enzyme that acts on all substrates ranging from 14:0- to 18: 1-ACP with predominant activity on 16:0-ACP. The substrate specificity profile of this enzyme is identical to that of the previously isolated enzyme, ChFatB1 (Jones et al. (1995) Plant Cell, 7:359-371). The in vitro hydrolytic activity of ChFatB3 shows a strong preference for 16:0-ACP with moderate activity on 14:0-ACP. These results are somewhat surprising since sequence analyses of ChFatB3 shows it is most homologous to C. palustris CpFatB2, a 14:0-ACP specific enzyme (Dehesh et al., (1996) Plant Physiol. 110:203-210). The in vitro hydrolytic activity of ChFatAl is similar to other class A TEs (Knutzon et al., (1992) Proc Natl Acad Sci USA 89:2624-2628), that act primarily on 18:1-ACP.

### EXAMPLE 3

The coding sequence of several TEs are placed under the control of T7 promoter from a Pet3a vector, resulting in construction of pCGN4865 (ChFatB1), pCGN4866 (ChFatB2), pCGN4867 (ChFatB3), pCGN4868 (ChFatAl), pCGN4869 (CpFatB1) and pCGN4870 (CpFatB2). For comparison, an SP6 vector containing the full-length precursor of small subunit of rubisco from pea (Olsen and Keegstra, (1992) J. Biol Chem. 267:433-439) is included in these studies. The polynucleotide molecules encoding the transit peptides of ChFatB2 (SEQ ID NO:12), ChFatB1 (SEQ ID NO:8), CpFatB2 (SEQ ID NO:2) and prSS the small subunit of pea rubisco (SEQ ID NO: 16) were also fused to the coding region of Green Flourescent Protein (GFP) resulting to the construction of pCGN8373, pCGN8374, pCGN8375 and pCGN8376, respectively. These plasmids were subsequently linearized with either Eco RV (ChFatB1 and CpFatB2), Nhe I (ChFatB2 and ChFatB3), Bam HI (ChFatAl) or Bgl II (CpFatB1). Uncapped mRNA was generated using Promega transcription protocol using either SP6 or T7 RNA polymerase. Radiolabeled protein was synthesized using nuclease-treated rabbit reticulocyte lysate and the standard reaction conditions as described by Promega with the following modification: translation reactions were incubated for 90 min at 25° C. as described by Bruce et al. (1994) In Plant Molecular Biology Manual, Volume 2 (Gelvin, S. B. and Schilperoort, R. B. eds), Kluwer Academic Publishers, Boston, pp1-15; henceforth referred to as Bruce et al., 1994.

All proteins were labeled with [³⁵S]-methionine (NEN, Boston, Mass.). Finally, all FatA and FatB class TE translation products were diluted with an equal volume of 50 mM cold methionine/import buffer, prior to use. Translated prSS was also diluted with an equal volume of 2 X import buffer containing 50 mM unlabeled methionine before use in a Pea chloroplast import assay.

### EXAMPLE 4

Intact chloroplasts are isolated from 8-12 day old pea seedlings (Pisum sativum var.

Little Marvel) by homogenization and differential centrifugation followed by sedimentation through a Percoll gradient as previously described (Bruce et al., 1994). Chloroplasts are washed twice in 50 mM HEPES-KOH (pH 7.7), 0.33 M Sorbitol (import buffer) and finally resuspended to a concentration of 1 mg of chlorophyll/ml of import buffer.

Import assays, with radiolabeled protein synthesized from nuclease-treated rabbit reticulocyte lysate, were performed as follows: Translation mixture (−5×10⁵ dpm) and ATP (4 mM final concentration) were added to chloroplasts (25 μg chlorophyll equivalent) in a final volume of 150μl and incubated at 25° C. for various times. Import assays were quenched by pelleting chloroplasts through a cushion of 40% Percoll (v/v) (Amersham Pharmacia Biotech, Piscataway, N.J.). The imported proteins were analyzed by SDS-PAGE and fluorography. Gels were quantitated directly by phosphornmager (Molecular Dynamics, Sunnyvale, Calif.).

Preparation of crude membrane fractions was as follows: after the indicated time, intact chloroplasts were reisolated by centrifugation through 40% Percoll. The recovered chloroplasts were lysed hypotonically on ice for 20 minutes. Fractions were recovered as described by Bruce et al., 1994 to yield a crude membrane and soluble fraction. Both membrane and soluble fractions were analyzed by SDS-PAGE and fluorography. Gels were quantitated directly by phosphornmager (Molecular Dynamics, Sunnyvale, Calif.).

For Na₂CO₃ extraction analysis of envelope membrane fraction, intact chloroplasts were re-isolated through a 40% Percoll cushion after a standard import reaction. The intact chloroplasts were lysed hypotonically and fractionated as described by Perry and Keegstra, (1994) Plant Cell 6:93-105) with the following modification: fractionation was performed with a sucrose step gradient consisting of 0.46 and 1.2 M sucrose solutions. The envelope membrane fraction located at the interface of 0.46/1.2M region was removed and re-isolated by ultracentrifugation. The soluble fraction was isolated by TCA precipitation. Half of the crude envelope fraction was extracted with 100 mM sodium carbonate as described by Fujiki et al., (1982a) J Cell Biol 93:97-102) and, (1982b) J Cell Biol 93:103-110) for 30 minutes on ice and then separated into envelope membrane and supernatant fractions by ultracentrifugation. The envelope membrane fraction was extracted again by resuspending in sodium carbonate and pelleting. Finally supernatants from both extractions were recovered by TCA precipitation. Both the extracted supernatant and envelope membrane were solubilized by sample buffer. All fractions were analyzed by SDS-PAGE and fluorography. Gels were quantitated directly by Phosphorlmager (Molecular Dynamics).

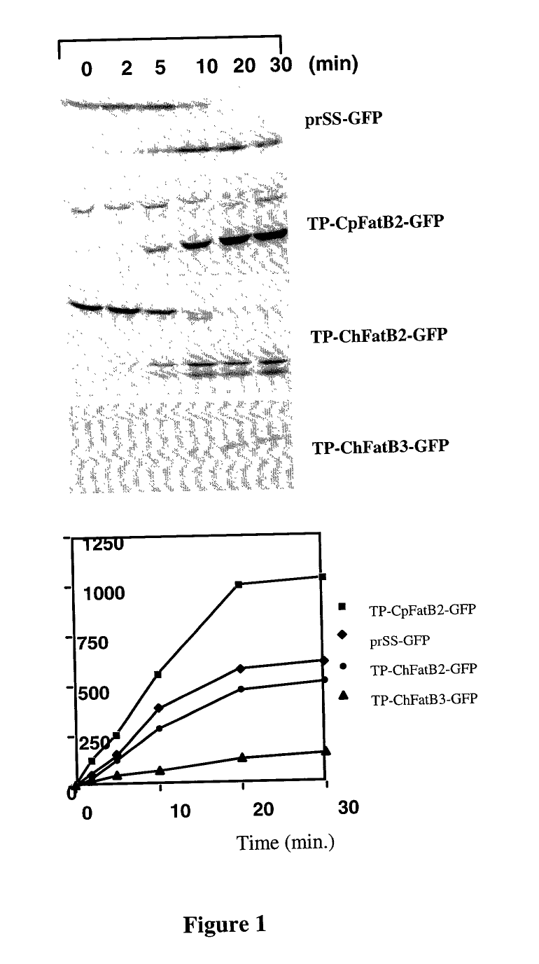

For comparison of transport efficacy, TP-GFP fusion proteins expressed from pCGN8373, pCGN8374, pCGN8375 and pCGN8376 were assayed in a Pea choroplast import assay. The TP-CpFatB2 transit peptide showed the highest rate and extent of import (measured by the amount of GFP molecules imported/chloroplast per minute) compared to prSS, ChFatB2 or ChFatB3 transit peptides fused to GFP (FIG. 1).

The results of the other import assays confirmed that Cuphea transit peptides could function specifically to target and transport heterologous proteins into pea chloroplasts. Comparing the difference in mobility between the precursor and the mature TE allowed a reasonable prediction of the size of the transit peptides (i.e., from 7-9 kDa in molecular mass). To further confirm that all Cuphea FatA and FatB TEs were imported into the chloroplasts, protease protection assays were performed with thermolysin. Thermolysin can not penetrate the plastidial envelope, therefore, proteins that have been imported into chloroplasts are protected and can not be digested by this protease. To determine the localization of FatA and FatB TEs within the chloroplasts, the import assays were separated into crude membrane and supernatant fractions. ChFatB2 was imported poorly into chloroplasts and fractionated exclusively to the membrane fractions, while ChFatB1 and CpFatB1 fractionated to both the membrane and the soluble fraction. When this dual localization was quantitated, approximately 80% of ChFatB1 associated with the membrane, while 20% resided in the supernatant. Similarly, approximately 75% of CpFatB1 associated with the membrane and 25% resided in the supernatant. ChFatAl, CpFatB2 and ChFatB3 all localized predominately to the supernatant fraction.

ChFatB1 and CpFatB1 were imported into pea chloroplasts and subsequently fractionated into envelope membrane (mixture of outer and inner membranes) and supernatant using a sucrose step gradient and centrifugation. This approach revealed that indeed a large portion of ChFatB1 and CpFatB1 associated with the chloroplastic envelope membrane. In the case of imported ChFatB1 approximately 78% associated with the envelope membrane and 22% localized to the supernatant fraction. Imported CpFatB1 showed a similar fractionation pattern, with 70% associating with the envelope membrane and 30% with the supernatant. When the portion of the ChFatB1 and CpFatB1 that associated with the envelope membrane was extracted with sodium carbonate (Fujiki et al., 1982a, 1982b ) the localization of ChFatB1 and CpFatB1 was altered. After sodium carbonate extraction, only 20% of ChFatB1 associated with the envelope membrane while 80% was extracted from the envelope fraction and became soluble. Cp FatB1 fractionated in a similar fashion after sodium carbonate extraction with 25% associating with the envelope membrane and 75% localized to the supernatant. These results suggest that both ChFatB1 and CpFatB1 are peripherally associated with the plastidial envelope membrane either directly or indirectly via other fatty acid biosynthetic enzymes associated with the plastidial envelope.

### EXAMPLE 5

Complementary DNAs (cDNAs) used for production of transgenic Arabidopsis were cloned into the seed specific expression cassette pCGN3223 (described in U.S. Pat. No. 5,639,790, the entirety of which is incorporated herein by reference), driven by a napin gene promoter, P-Napin (Kridl et aL, (1991) Seed Sci Res. 1:209-219). To fuse the TP-CpFatB2 transit peptide to the mature portion of ChFatB2 to make TP-CpFatB2-ChFatB2, the following oligonucleotides were generated:

ChFatB2 5′: GAATTCTGGCCAGACATGCATGATCGGAAATCCAAG (SEQ ID NO:29),

ChFatB2 3′:GAATTCTCTAGAGTACCAGATCTCTAAGAGACCGAGTTTCCATTTGAA

GTCTTTCCCGTTGAT (SEQ ID NO:30).

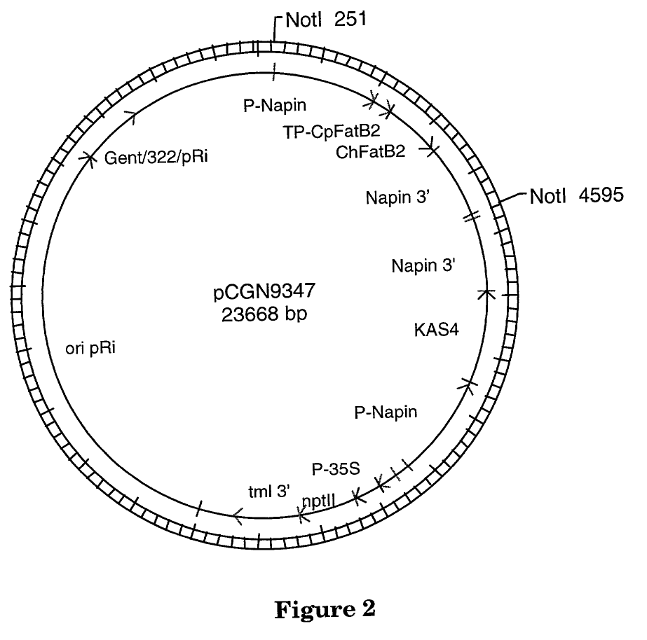

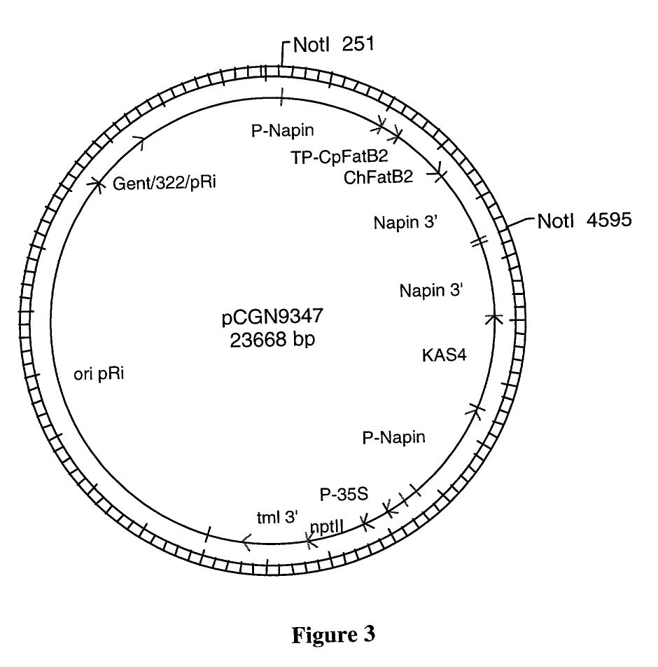

The cDNA portion of CpFatB2 clone encoding the mature polypeptide (Dehesh et al., (1996) Plant Physiol. 110:203-210) was removed by a BalI and XhoI digest. This plasrnid was subsequently used for the insertion of the ChFatB2 PCR product digested with compatible enzymes, leading to construction of the chimera (TP-CpFatB2-Ch FatB2). Followed by a Smal and BglII digest the TP-CpFatB2-ChFatB2 insert was isolated and introduced into Sall, filled, BglII linearized PCGN3223 plasmid. Subsequently the P-Napin driven Ch FatB2 either with its native (ChICh FatB2) transit peptide (Dehesh et al (1996) Plant J. 9:167-172), or TP-CpFatB2 transit peptide, was introduced into the NotI site of pCGN5401, the binary vector containing the ChKAS4 cDNA (Dehesh et al., (1998) Plant J. 15(3):383-390), resulting to the construction of pCGN9346 (FIG. 2) and pCGN9347 (FIG. 3), respectively. These two binary constructs were used to transform Arabidopsis thaliana. Transformation of Arabidopsis was by vacuum infiltration (Bechtold et al., (1993) CR. Acad. Sci. 316:1194-1199).

### EXAMPLE 6

The efficient plastidial targeting property of the CpFatB2 transit peptide (SEQ ID NO: 1 and the deduced amino acid sequence provided in SEQ ID NO:2) for improving the quantity and compositions of transgene phenotypes, such as that of particular fatty acid production, for example, of medium chain fatty acids in transgenic seeds was demonstrated in Arabidopsis plants transformed with ChFatB2 acyl-ACP thioesterase fused to CpFatB2 transit peptide (TP-CpFatB2-ChFatB2). For direct comparison the native ChFatB2 acyl-ACP thioesterase (TP-ChFatB2-ChFatB2) was also introduced into Arabidopsis. It was previously shown by Dehesh et al., (1998) Plant J. 15(3):383-390); and Leonard et al, (1998) Plant J. 13:612-628, that maximum accumulation of medium-chain fatty acids in seed oil is achieved upon the co-expression of a medium-chain TE with KAS4 (also described in PCT Publication WO 98/46776, the entirety of which is incorporated herein by reference), a medium chain specific condensing enzyme. Therefore, to obtain high levels of these fatty acids, Arabidopsis plants were transformed with a binary vector containing Ch KAS4 (Dehesh et al., (1998) Plant J. 15(3):383-390) in tandem plant expression cassettes with either the native nChFatB2 (native transit peptide and coding sequence of ChFatB2, pCGN9346) or TP-CpFatB2-ChFatB2 (chimeric coding sequence of the transit peptide of CpFatB2 fused with ChFatB2, pCGN9347). Transgenic plants were grown simultaneously under the same environmental conditions. Total fatty acid composition of T2 seeds obtained from a minimum of 20 independent primary transformants expressing either pCGN9346 or pCGN9347 were analyzed.

The quantities and composition of triglyceride fractions from reverse-phase HPLC were determined by acidic methyl esters essentially according to the method of Browse et al., (1986) Anal. Biochem. 152:141-152. Tri-17:0 triglyceride was included as an internal standard. Based on these analyses, a difference between the oil composition of these two groups of transgenic seeds, was in the levels of 8:0 and 10:0 in their oil (Table 1). The total levels of 8:0 and 10:0 fatty acids across all pCGN9347 transgenic lines were higher on average (12.47), as well as for the maximum(16.12) and minimum (7.72) levels, than the respective counterparts in the pCGN9346 containing transgenic lines. These data clearly show that the TP-CpFatB2 enhanced accumulation of medium chain fatty acids in transgenic Arabidopsis seeds and provided a modified oil content of the oil extracted from the seeds.

TABLE 1
Percent Levels of 8:0 + 10:0 Fatty Acids from Arabidopsis seeds
transgenic for pCGN9346 and pCGN9347

| pCGN9346 Line# | 8:0 + 10:0 | pCGN9347 Line# | 8:0 + 10:0 |
|---|---|---|---|
| AT00014-8 | 1.37 | AT00011-10 | 7.72 |
| AT00014-2 | 3.14 | AT00011-7 | 8.39 |
| AT00014-1 | 5.4 | AT00011-13 | 9.98 |
| AT00014-10 | 5.7 | AT00011-2 | 10.29 |
| AT000l4-18 | 6.31 | AT000l1-14 | 11.46 |
| AT00014-7 | 6.61 | AT00011-18 | 11.68 |
| AT00014-30 | 6.9 | AT00011-17 | 11.82 |
| AT00014-12 | 7.13 | AT00011-3 | 12.0 |
| AT00014-16 | 7.17 | AT00011-1 | 12.3 |
| AT00014-13 | 7.36 | AT00011-12 | 12.77 |
| AT00014-28 | 7.49 | AT00011-5 | 12.79 |
| AT00014-5 | 7.64 | AT00011-16 | 12.79 |
| AT00014-14 | 7.65 | AT00011-9 | 12.83 |
| AT00014-22 | 7.99 | AT00011-19 | 13.26 |
| AT00014-24 | 8.03 | AT0G011-11 | 13.62 |
| AT00014-17 | 8.26 | AT00011-20 | 13.81 |
| AT00014-29 | 8.47 | AT00011-15 | 14.66 |
| AT00014-19 | 8.54 | AT00011-4 | 15.14 |
| AT00014-27 | 8.55 | AT00011-8 | 15.99 |
| AT00014-11 | 8.91 | AT00011-6 | 16.12 |
| AT00014-25 | 9.06 | | |
| AT00014-6 | 9.2 | | |
| AT00014-9 | 9.81 | | |
| AT00014-15 | 10.5 | | |
| AT00014-3 | 10.56 | | |
| AT00014-4 | 11.11 | | |
| AT00014-26 | 13.32 | | |

The above results demonstrate that improved efficiency of plastid importation of protein sequences derived form DNA sequences of interest may be obtained using the plastid transit peptides sequences of the present invention. The plastid transit peptide sequences find use in the preparation of expression constructs to provide chloroplast and seed plastid targeting for a wide variety of genes involved in plant genetic engineering applications. Furthermore, the sequences of the present invention find use in the enhancement of traits introduced into a plant cell.

All publications and patent applications mentioned in this specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claim.

What is claimed is:

1. A recombinant DNA construct comprising: a promoter molecule that functions in plant cells, operably linked to a heterologous DNA molecule encoding a plastid transit peptide of a Cuphea acyl-ACP thioesterase, operably linked to a heterologous DNA molecule encoding a protein, operably linked to a DNA molecule providing 3′ termination functions.

2. A recombinant DNA construct of claim 1, wherein said plastid transit peptide comprises a peptide sequence with at least 90% identity to peptide sequences selected from the group consisting of SEQ ID NO:2, SEQ ID NO:4, SEQ ID NO:6, SEQ ID NO:8, SEQ ID NO: 10, SEQ ID NO:12, and SEQ ID NO:14

3. A recombinant DNA construct of claim 1, wherein said heterologous DNA molecule encoding a plastid transit peptide of a Cuphea plant acyl-ACP thioesterase comprises a nucleotide sequence with at least 60% identity to DNA molecules selected from the group consisting of SEQ ID NO: 1, SEQ ID NO:3, SEQ ID NO:5, SEQ ID NO:7, SEQ ID NO:9, SEQ ID NO: 11, and SEQ ID NO: 13.

4. A recombinant DNA construct comprising: a promoter molecule that functions in plant cells, operably linked to a heterologous DNA molecule encoding a plastid transit peptide with at least 90% identity to SEQ ID NO:2, operably linked to a heterologous DNA molecule encoding a protein, operably linked to a DNA molecule providing 3′ termination functions.

5. A recombinant DNA construct of claim 4, wherein said heterologous DNA molecule encoding a plastid transit peptide comprises a nucleotide sequence at least 60% homologous to SEQ ID NO:1.

6. A recombinant DNA construct of claim 4, wherein said protein confers agronomically desirable traits selected from the group consisting of herbicide tolerance, insect resistance, stress resistance, disease resistance, high oil production, modified oil production, high starch production, high protein production, high yield, enhanced nutrition, enhanced processing, pharmaceutical peptides, transgenic plant identification, and enhanced storage life.

7. In a method for the translocation of a protein to a crop plant cell plastid, the improvement comprising introducing into a crop plant cell a recombinant DNA construct of claim 4.

8. In the method of claim 7, a further step comprising regenerating said crop plant cell into a transgenic crop plant.

9. A recombinant DNA construct comprising: a promoter molecule that functions in plant cells, operably linked to a heterologous DNA molecule encoding a plastid transit peptide of a Cuphea palustris FatB2 acyl-ACP thioesterase, operably linked to a heterologous DNA molecule encoding an acyl-ACP thioesterase, operably linked to a DNA molecule providing 3′ termination functions.

10. A recombinant DNA construct of claim 9, wherein said plastid transit peptide comprises a peptide sequence with at least 90% identity to SEQ ID NO:2.

11. A transgenic crop plant comprising the recombinant DNA construct of claim 10.

12. A method for producing a modified fatty acid composition of an oilseed crop comprising: a) transforming a plant cell of an oil seed crop with the recombinant DNA construct of claim 10 and a DNA construct that provides expression of a medium chain specific condensing enzyme; b) regenerating the plant cell into a transgenic oil seed crop plant; c) planting seeds of said transgenic oil seed crop plant; d) harvesting seeds from said transgenic oil seed crop plant; e) processing said seeds for purification of a modified oil content.

13. A modified oil content produced by the method of claim 12.
